SEQUENCE LISTING (4 sequences)

SEQ ID NO: 1 - protein, 456 aa, Homo sapiens
MRKSPGLSDCLWAWILLLSTLTGRSYGQPSLQDELKDNTTVFTRILDRLLDGYDNRLRPGLGERVTEVKTDIFVTSFGPVSDHDMEYTIDVFFRQSWKDERLKFKGPMTVLRLNNLMASKIWTPDTFFHNGKKSVAHNMTMPNKLLRITEDGTLLYTMRLTVRAECPMHLEDFPMDAHACPLKFGSYAYTRAEVVYEWTREPARSVVVAEDGSRLNQYDLLGQTVDSGIVQSSTGEYVVMTTHFHLKRKIGYFVIQTYLPCIMTVILSQVSFWLNRESVPARTVFGVTTVLTMTTLSISARNSLPKVAYATAMDWFIAVCYAFVFSALIEFATVNYFTKRGYAWDGKSVVPEKPKKVKDPLIKKNNTYAPTATSYTPNLARGDPGLATIAKSATIEPKEVKPETKPPEPKKTFNSVSKIDRLSRIAFPLLFGIFNLVYWATYLNREPQLKAPTPHQ

SEQ ID NO: 2 - protein, 844 aa, Meriones unguiculatus
MGPGAPCPPVGWPLPLLLVIAAGMAPVWASHSPHLPRPHPRVPPHPSSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKTYDPSINCTVDEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDVLSWTKTDKWIGGSPPADQTLVIKTFRFLSQKLFISVSVLSSLGIVLAVVCLSFNIYNSHVRYIQNSQPNLNNLTAVGCSLALAAVFPLGLDGYHIGKSQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSKKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSEAQDTMKTGSSTNNNEEEKSRLLEKENRELEKIIAEKEERVSELRHQLQSRQQLRSRRHPPTPPDPSGGLPRGPSEPPDRLSCDGSRVHLLYK

SEQ ID NO: 3 - DNA, 2535 bp, Meriones unguiculatus
  source 1..2535: mol_type = other DNA
atgggcccggggggaccctgtaccccggtggggtggccgctgcctcttctgctggtgatggcggctggggtggctccggtgtgggcctctcactcccctcatctcccgcggcctcacccgagggtccccccgcacccctcctcagaacggcgtgcagtgtacatcggggcgctgtttcccatgagcgggggctggccggggggccaggcctgccagcccgcggtggagatggcgctggaggacgtgaacagccgcagggacatattgccggactacgagctcaagcttatccaccacgacagcaagtgtgacccagggcaagccaccaagtacttgtatgaactactctacaatgaccccatcaagattatcctcatgcctggctgcagttctgtctccacacttgtggctgaggctgcccggatgtggaacctaattgtgctctcatatggctccagctcaccagctttgtcaaaccgacagcgttttccaacattcttccgaacacatccgtctgccacactccacaatcccacccgggtgaaactctttgaaaagtggggctggaagaagattgccaccatccagcagaccacagaggtcttcacttcaactctggatgacctagaggagcgagtgaaggaggctgggattgagatcacgttccgacagagtttcttctcagatccagctgtgcctgttaaaaacctgaagcgtcaggatgctcgaatcatcgtgggacttttctatgagactgaagcccggaaagttttttgtgaggtctataaggaacggctctttgggaagaagtatgtctggttcctcattgggtggtatgctgacaactggttcaagacctatgacccatcaatcaactgtacagtggatgaaatgaccgaggcagtggaaggccatatcaccacggagatcgtcatgctgaaccctgccaacacccgaagcatttccaacatgacatcccaggaatttgtggagaaactaaccaagaggctgaagagacaccccgaggaaactggaggcttccaggaggcaccactggcctatgatgctatatgggccttggctctggccttgaataagacctctggaggaggtggccgctcgggtgtgcgcctggaggactttaactacaacaatcagaccattacagaccaaatctaccgggccatgaactcctcctcctttgagggtgtttctggccatgtggtctttgatgccagcggctcccggatggcatggacacttattgagcagttacagggcggcagctacaagaagattggctactacgacagcaccaaggatgttctctcctggaccaaaacagataagtggattggagggtctcctccagctgaccagaccttggtcatcaagacattccgtttcctgtcacagaaactctttatctccgtctcagttctctccagcctgggcattgttcttgctgttgtctgtctgtcctttaacatctacaactcccacgttcgttatatccagaactctcagcccaacctgaacaatctgactgccgtgggctgctcactggcactagctgctgtctttccccttgggctggatggttaccacatagggaaaagccagttcccatttgtctgccaggcccgactttggctcttgggcttgggctttagtctgggttatggctctatgttcaccaagatctggtgggtccacacagtcttcacaaagaaggaggagaagaaggaatggaggaagaccctagaaccttggaaactgtatgccactgtaggcttgctggtgggaatggatgtcctgactcttgccatctggcagattgtggaccccttgcaccgaaccattgagacttttgccaaggaggagccaaaggaagacattgatgtctccatcctgccccagctggagcactgcagctccaagaagatgaatacatggcttggcattttctatggttacaaggggctgctgctgctgctgggaatctttcttgcctatgagactaaaagcgtgtccactgaaaagatcaacgaccacagggctgtgggtatggccatctacaatgttgcggtcctgtgtctcatcactgctcctgtgaccatgattctttccagccagcaggacgcagccttcgccttcgcctctctggccatcgtgttctcttcctacattacgctggttgtgctctttgtgcccaagatgcgcaggttgatcactcggggggagtggcagtccgaagcgcaagacaccatgaaaacggggtcgtccaccaacaacaacgaggaagagaagtcccggctgttggagaaagaaaaccgtgaattggaaaagatcattgctgagaaagaggagcgggtctcggaactgcgccatcagctccagtctcggcagcagctgcgctcacggcgccaccctccaacacccccagacccctctgggggccttcccaggggcccctcggagccccctgaccggctgagctgtgatgggagtcgagtgcatttgctgtacaagtga

SEQ ID NO: 4 - protein, 844 aa, Homo sapiens
MGPGAPFARVGWPLPLLVVMAAGVAPVWASHSPHLPRPHSRVPPHPSSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKIYDPSINCTVDEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIGGSPPADQTLVIKTFRFLSQKLFISVSVLSSLGIVLAVVCLSFNIYNSHVRYIQNSQPNLNNLTAVGCSLALAAVFPLGLDGYHIGRNQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSRKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSEAQDTMKTGSSTNNNEEEKSRLLEKENRELEKIIAEKEERVSELRHQLQSRQQLRSRRHPPTPPEPSGGLPRGPPEPPDRLSCDGSRVHLLYK

(19) United States
(12) Patent Application Publication
(10) Pub. No.: US 2025/0352673 A1
(43) Pub. Date: Nov. 20, 2025

(54) COMPOSITIONS AND METHODS FOR TREATING SEQUELAE OF HEARING LOSS

(71) Applicant: New York University, New York, NY (US)

(72) Inventors: Dan SANES, New York, NY (US); Samer MASRI, Brooklyn, NY (US)

(21) Appl. No.: 18/880,932

(22) PCT Filed: Jul. 7, 2023
(86) PCT No.: PCT/US2023/069786

§ 371 Date: Jan. 3, 2025

Related U.S. Application Data
(60) Provisional application No. 63/359,724, filed on Jul. 8, 2022.

(51) Int. Cl. A61K 48/00; A61K 9/00; A61K 38/17; A61P 27/16; C12N 15/86

(52) U.S. Cl. CPC A61K 48/0058; A61K 9/0048; A61K 38/1787; A61K 48/0075; A61P 27/16; C12N 15/86; C12N 2750/14143

(57) ABSTRACT

Provided are compositions and method for prophylaxis and/or therapy of hearing loss or related dysfunctions, including but not limited to tinnitus, that could be ameliorated by restoring central nervous system inhibitory synapses. The compositions include polynucleotides and viral vectors that are used to express at least one GABA receptor component which may be a GABA_{A} receptor alpha 1 subunit or GABAR receptor Ib subunit. Expression of the GABA receptor may be under control of a CaMKII promoter.

### CROSS REFERENCE TO RELATED APPLICATION

This application claims priority to U.S. provisional application No. 63/359,724, filed Jul. 8, 2022, the entire disclosure of which is incorporated herein by reference.

### STATEMENT REGARDING FEDERALLY SPONSORED RESEARCH

This invention was made with government support under grant no. R01 DC011284 awarded by the National Institutes of Health. The government has certain rights in the invention.

### SEQUENCE LISTING

The instant application contains a Sequence Listing which is submitted in.xml format and is hereby incorporated by reference in its entirety. Said.xml file is named “058636_00621_ST26.xml”, was created on Jul. 7, 2023, and is 9,018 bytes in size.

### FIELD

The present disclosure relates generally to prophylaxis and/or therapy of hearing loss, and more specifically to use of recombinant polynucleotides and viral vectors with a sequence encoding a Gamma-aminobutyric acid (GABA) receptor.

### BACKGROUND OF THE DISCLOSURE

Many developmental disorders are associated with a reduction in the strength of inhibitory synapses, especially those that use the amino acid neurotransmitter GABA. Specifically, there is evidence that GABA receptors are reduced in the postsynaptic membrane, thereby leading to smaller inhibitory synaptic currents. This effect is especially profound for developmental sensory deprivation, such as that occurring with congenital hearing loss or blindness. In fact, a dramatic decrease in GABA receptors occurs even when developmental hearing loss is temporary, such as that occurring during middle ear infections. The reduction in the strength of GABAergic inhibitory synapses is commonly associated with behavioral deficits. Thus, it has been demonstrated that hearing loss causes a decrease in the strength of inhibitory synapses in auditory cortex, and this is accompanied by diminished performance on auditory perceptual tasks. In humans, developmental hearing loss is commonly accompanied by deficits in speech and language acquisition. Thus, there is an ongoing and unmet need to form compositions and methods that can increase inhibition after hearing loss to restore normal neural function. The present disclosure is pertinent to this need.

### BRIEF SUMMARY OF THE DISCLOSURE

The present disclosure relates to compositions and methods for use in prophylaxis and/or therapy of hearing loss-induced deficits. Aspects of the disclosure relate in part to the surprising demonstration that recombinant expression of a GABA receptor subunit in certain neurons is sufficient to improve auditory perceptual skills that are associated with hearing loss.

The disclosure provides polynucleotides encoding a GABA receptor subunit.

The sequence encoding the GABA receptor subunit may be provided in the form of a viral vector, illustrated using a recombinant adeno associated virus (rAVV) vector. The sequence encoding the GABA receptor subunit operably linked to a suitable promoter, illustrated using a CaMKII0.4 promoter. The encoded GABA receptor subunits include GABA_{B} and GABA_{A} subunits.

Embodiments of the disclosure are demonstrated by administering to an individual an rAAV comprising: i) a CaMKII0.4 promoter sequence, and ii) a sequence encoding the GABA_{B} receptor subunit, also referred to herein as the “1b” subunit and as “Gabrb1b,” The Gabrb1b is expressed in pyramidal neurons of the individual. Thus, in one aspect the disclosure provides for modifying pyramidal neurons in an individual to increase expression of Gabrb1b in Gabrb1b-expressing neurons. Using the GABA_{B} subunit as proof of principal, the disclosure includes a similar approach using polynucleotides and expression vectors that encode a GABA_{A} subunit.

### BRIEF DESCRIPTION OF THE FIGURES

FIG. 1. Illustration depicting the two classes of GABA receptor. GABA_{A} receptors are composed of 5 subunits, one of which we express, the alpha 1 subunit. GABA_{B} receptors are composed to two subunits, one of which we express, the 1b subunit.

FIG. 2. Schematic depiction of core pathology that occurs following developmental hearing loss and other developmental disorders, attributable to a reduction of GABA-mediated inhibitory synaptic potentials. The right portion of the schematic shows a loss of both GABA_{A} and GABA_{B} receptor-mediated potentials, as recorded from the postsynaptic neuron (control is shown for comparison, dashed gray trace).

FIG. 3 provides a schematic depiction of representative differences between traditional pharmaceutical approaches and an embodiment of the present disclosure. The relative disadvantages of the pharmacological approach and advantages of the disclosure are listed below each schematic.

FIG. 4. Photograph of anatomical evidence for expression of Gabrblb using a representative viral construct of the disclosure, along with a reporter protein in the form of Turbo Red Fluorescent Protein (TurboRFP) using the viral construct AAVI.CaMKII0.4.Gabrb 1b.P2A. TurboRFP. WPRE3.rBG. The co-expressed fluorophore, TurboRFP, was expressed at the injection site. Panel A shows a lateral view of the whole brain. Even under normal lighting conditions, TurboRFP is visible (dotted circle). Panel B shows a transverse tissue section from the brain shown in panel A. TurboRFP fluorescence is visible throughout the auditory cortex (AC) following 3 weeks of expression.

FIG. 5 shows a schematic time line for induction of temporary hearing loss from postnatal days 10-23, the virus manipulation (using representative AAV-Gabrb1b or control virus), and the outcome measures (behavior and physiology to confirm expression). An amplitude modulated stimulus is illustrated in the inset. Large negative values signify smaller modulations that are more difficult to detect in behavioral tests.

FIG. 6 provides graphs summarizing behavioral results from an experiment and demonstrate that developmental hearing loss impairs a perceptual skill (amplitude modulation detection). FIG. 6 also shows a representative construct expressing Gabrb1b can rescue the hearing loss-induced deficit.

FIG. 7 provides a graph summarizing behavioral results shows the behavioral results from an experiment and demonstrate that developmental hearing loss impairs a second perceptual skill (spectral modulation detection), and that a representative construct expressing Gabrb 1b can rescue the hearing loss-induced deficit.

FIG. 8. Diagram (A) showing for both Gabral and Gabbr1b AAVs, primary auditory cortex layer ⅔ was injected. After three weeks a thalamocortical slice preparation was made and whole cell recordings (current clamp) from ACx L⅔ pyramidal cells were carried out. (B) Top, diagram showing Gabral vector. Bottom, micrograph from ACx L⅔ showing Gabral infected cells (mCherry) and one patched pyramidal neuron. (C) Representative evoked IPSP showing the larger GABA_{A} potential in the Gabral infected pyramidal neuron (fluorescing patched cell from B) vs local uninfected (non-fluorescing) pyramidal neuron from the same slice. D) Plot diagram showing the average difference in GABA_{A} IPSP amplitudes for uninfected versus Gabral infected pyramidal neurons. (E) Top, Diagram showing Gabbr 1b vector. Bottom, micrograph from AC L⅔ showing Gabbr1b infected cells (turboRFP) and one patched pyramidal neuron. (F) Representative evoked IPSP showing the larger GABA_{B} potential in the Gabbr1b infected pyramidal neuron (from C) vs local uninfected (non-fluorescing) pyramidal neuron from the same slice. (G) Plot diagram showing the average difference in GABA_{B} IPSP amplitudes for uninfected versus Gabbr1b infected pyramidal neurons.

FIG. 9 provides a schematic of a vector used in the Examples of this disclosure that is referred to as AAV1.CaMKII0.4.Gabral.IRES.mCherry.WPRE.rBG.

### DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein, and every polynucleotide sequence referred to herein includes its complementary DNA sequence, and also includes the RNA equivalents thereof to the extent an RNA sequence is not given. Every DNA and RNA sequence encoding polypeptides disclosed herein is encompassed by this disclosure. All amino acid sequences encoded by polynucleotides described herein are included in the disclosure. The disclosure includes all polynucleotide sequences that encode all amino acid sequences described herein. Any sequence referred to by a database entry is incorporated herein by reference as the sequence exists in the database as of the effective filing date of this application or patent. Amino acid and polynucleotide sequences having at least 95% identity to the sequences provided here are included in the disclosure. All of the amino acid sequences described herein can include amino acid substitutions, such as conservative substitutions, that do not adversely affect the function of the protein that comprises the amino acid sequences. The sequences of this disclosure may comprise or consist of the described sequences.

The present disclosure comprises compositions and method for prophylaxis and/or therapy of hearing loss or related dysfunctions, including but not limited to tinnitus, that could be ameliorated by restoring central nervous system inhibitory synapses. In an embodiment, use of a described composition may specifically increase inhibition at the region of the brain coding for the experienced tinnitus frequency.

In embodiments, the individual has amblyopia, developmental cataracts, or another disorder related to sensory perception. Thus, in embodiments, the disclosure relates to prophylaxis and/or therapy of central nervous system sequelae of hearing loss. The disclosure provides recombinant polynucleotides and viral vectors that are used to express at least one GABA receptor component. The expressed GABA receptor may comprises a GABA_{A} receptor alpha 1 subunit or GABA_{B} receptor 1b subunit. Single polynucleotides and viral vectors encoding only one of these receptors, combinations of polynucleotides encoding both receptor types, and single polynucleotides encoding both receptors, and uses thereof are encompassed by the disclosure. As an alternative to expressing the GABA receptor(s) in cells of an individual, the disclosure includes introducing the described polynucleotides into one or more chromosomes using, for example, guide-directed RNA nucleases, TALONS, zinc fingers, transposon-bases systems, and other designer nucleases that will be apparent to those skilled in the art given the benefit of this disclosure.

Results described in the figures and discussed further below were unexpected. First, the central nervous system sequelae of peripheral hearing loss are numerous. For example, molecular changes to excitatory synapses, neural membrane properties, and inhibitory synapses have all been documented for several central auditory structures, including cortex. Therefore, prior to the present disclosure, it was not expected that treating any single molecular deficit would lead to an improvement in auditory perception. Further, prior to the present disclosure, and without intending to be bound by any particular theory, it is considered that there was no direct evidence linking the strength of inhibitory synapses in auditory cortex with a normal perceptual skill. Furthermore, there was no direct evidence that expression of the GABA_{A} or GABA_{B} receptor under the CaMKII0.4 promotor would be therapeutic. While previous work suggests a potential reduction in the strength of inhibitory synapses can be correlated with changes to auditory cortex neuron sound-evoked responses, a causal link to any specific auditory behavior has remained theoretical. In contrast to this prior understanding, the present disclosure demonstrates that expression of a described GABA receptor is alone sufficient to improve or restore perceptual skills that are characteristic of normal hearing.

Certain examples and reductions to practice described herein were developed using the described gerbil models. Amino acid and nucleotide sequences of the described viral constructs that were used to produce the results are provided below. Based on the analysis using gerbils, it is expected that the same approach can be used with other mammals, including but not necessarily limited to humans. Amino acid and nucleotide sequences of construct components are provided below. The disclosure includes all amino acid and nucleotide sequences that are at least 80% similar to the described sequences across their entire lengths, provided the similar sequences retain their described function.

The construct used to express the GABA_{A} receptor alpha 1 subunit or GABA_{B} receptor 1b subunit, either alone or in combination, is configured such that expression of the subunit(s) is driven by a promoter that is operably linked to the receptor subunit coding sequence(s) By “operably linked” it is meant that the promoter sequence is present in the same polynucleotide as the sequence encoding the GABA receptor component (and is thus provided in cis), and expression of the GABA receptor is dependent on the presence and function of the promoter to promote transcription. The disclosure is illustrated using a mouse o-calcium/calmodulin-dependent protein kinase II (α-CaMKII) promoter, but other promoters may be used, including but not limited to the human α-CaMKII promoter. In embodiments, the promoter may be preferentially functional or exclusively functional in a particular anatomy, cell, or tissue type. In embodiments, the promoter is functional in auditory cortex neurons, presynaptic terminals, postsynaptic neurons, or both. In certain embodiments, a promoter for use in expressing a GABA_{B} 1a subunit is functional in presynaptic terminals. In certain embodiments, a promoter for use in expression a GABA_{B}1b subunit is functional in postsynaptic neurons

In embodiments, a viral vector is used to introduce the described polynucleotides into an individual. In an embodiment, the viral vector comprises a retroviral vector, such as a lentiviral vector. In embodiments, the viral vector comprises a recombinant adeno-associated virus (rAAV). In one embodiment, the viral vector comprised a self-complementary adeno-associated virus (scAAV).

Methods of this disclosure comprise introducing the modified rAAVs into neuronal cells in the brain of an individual in need thereof. Non-limiting embodiments of this disclosure are demonstrated in gerbils to demonstrate effects on sound perception. However, and as will be recognized by those skilled in the art, because the present disclosure includes a method for modifying neurons so that they comprise the described features, it is feasible for the present disclosure to have additional therapeutic applications that extend beyond hearing loss.

Suitable vectors that can be adapted to comprise a suitable promoter and encode the GABA receptor component, given the benefit of the present disclosure, are commercially available from, for example, the CLONTECH division of TAKARA BIO. In certain implementations plasmid vectors may encode all or some of the well-known rep, cap and adeno-helper components. The rep component comprises four overlapping genes encoding Rep proteins required for the AAV life cycle (Rep78, Rep68, Rep52 and Rep40). The cap component comprises overlapping nucleotide sequences of capsid proteins VP1, VP2 and VP3, which interact together to form a capsid of an icosahedral symmetry. Another plasmid providing the Adeno Helper function may also be co-transfected. The helper components comprise the adenoviral genes E2A, E4orf6, and VA RNAs for viral replication.

In embodiments, the polynucleotides encoding the GABA component in the rAAV can be modified, for example, by including optimized codons for expression in human neurons.

The described polynucleotides and vectors comprising the polynucleotides encode at least one GABA receptor subunit, and comprise a promoter that is operably linked to the GABA receptor subunit coding sequences. Other optional features of the vectors are illustrated in the accompanying figures and the description below. In one embodiment, such as for therapeutic purposes, the polynucleotide can be free from any sequence encoding a reporter protein. Likewise, components of the polynucleotides that are included for the purpose of expressing a reporter protein and visualizing the location of expression may be excluded from polynucleotides that are intended for therapeutic approaches. However, polynucleotides of this disclosure can comprise additional elements that will be apparent to those skilled in the art, given the benefit of the present disclosure.

In certain examples, the polynucleotides comprise a sequence encoding an element such as a Woodchuck hepatitis virus Posttrascriptional Regulatory Element (WPRE) or a variant thereof, which is believed to increase RNA stability and protein yield. The polynucleotides may also comprise a polyadenylation signal such as bovine growth hormone polyadenylation signal and/or SV40 polyomavirus simian virus 40 polyadenylation signal. The polynucleotide can comprise a minimal promoter, such as a human beta-globin minimal promoter (phβg) and a chimeric intron sequence. Without intending to be constrained by any particular theory it is considered that described rAVV vectors aid in concatamer formation in the nucleus after the single-stranded vector DNA is converted by host cell DNA polymerase complexes into double-stranded DNA. It is accordingly believed that administration of the rAAVs of this disclosure will form episomal concatemers in the nucleus of cells into which they are introduced. In non-dividing cells, such as adult neurons, it is believed these concatemers remain intact for the life of the neurons. It is also expected that integration of rAAV polynucleotides into host chromosomes will be negligible or absent and will not affect expression of regulation of any other human gene.

In various embodiments, the disclosure includes isolated and/or recombinant polynucleotides comprising a suitable promoter and the sequence encoding the GABA receptor subunit, expression vectors comprising such polynucleotides, cells comprising the polynucleotides, cells comprising rAAV encoded by the polynucleotides, isolated preparations of such rAAV particles, and pharmaceutical preparations comprising the rAAV particles.

In various aspects of the invention, methods of making the rAA Vs are provided. In general, the method of making the rAAvs comprises culturing cells which comprise an expression vector encoding an rAAV of this disclosure, allowing expression of the polynucleotides to produce the rAAVs, and separating the rAAVs from cells in the cell culture and/or from the cell culture media. The rAAVs can be purified to any desired degree of purity using conventional approaches.

rAAVs of the invention can be mixed with any pharmaceutically acceptable buffer, excipient, carrier and the like to form a pharmaceutical preparation. Suitable pharmaceutical compositions can be prepared by mixing rAAVs with a pharmaceutically-acceptable carrier, diluent or excipient, and suitable such components are well known in the art. Some examples of such carriers, diluents and excipients can be found in: Remington: The Science and Practice of Pharmacy (2022) 23rd Edition, Philadelphia, PA.

In general, a composition comprising a rAAV can be administered to any individual in need thereof. In embodiments, the individual is suffering from hearing loss, has suffered hearing loss, or is at risk for progression of hearing loss. In embodiments, the individual has been diagnosed with or is suspected of having peripheral hearing loss (i.e., either temporary or permanent dysfunction of the middle ear bones or the cochlea). In embodiments, the individual is suffering from an analogous sensory disorder, such as cataracts or amblyopia.

The rAAV can be administered using any suitable approach, such as intracranial injection, intravenous injection, or intranasal administration. In embodiments, the described rAAVs can be administered such that they enter and express the GABA receptor components at least in cortical pyramidal neurons.

In embodiments, the disclosure includes administering a therapeutically effective amount of an rAAV to an individual. “Therapeutically effective amount” as used herein means that amount of rAVV that is introduced into a sufficient number of neurons such that hearing loss-induced deficits are inhibited, or reversed. The amount of rAAV that is administered can be determined by those skilled in the art, given the benefit of the present disclosure and based on factors such as the size of the individual, age, gender, type, and severity of hearing loss. In embodiments, a therapeutically effective amount is an amount sufficient such that the sequelae of hearing loss in the individual is inhibited, or hearing of the individual is improved. In embodiments, a therapeutically effective amount is sufficient such that degenerative changes within the central nervous system that are induced by peripheral hearing loss are inhibited or prevented. In an embodiment, inhibiting one central sequela of hearing loss that comprises a reduction of auditory cortex synaptic inhibition mediated by GABA_{A} and/or GABA_{B} receptors. In an embodiment, the disclosure provides for recovery of normal performance on auditory perceptual tasks, non-limiting examples of which are described below. In embodiments, comprehension of human speech is improved.

The approaches of the present disclosure can also be combined with other anti-hearing loss techniques, including but not necessarily limited to use with therapeutic agents, and/or medical devices.

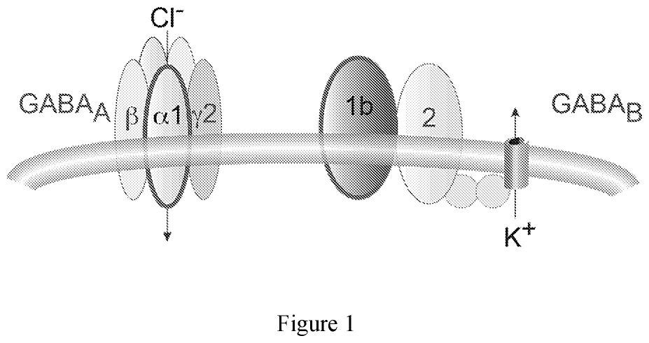

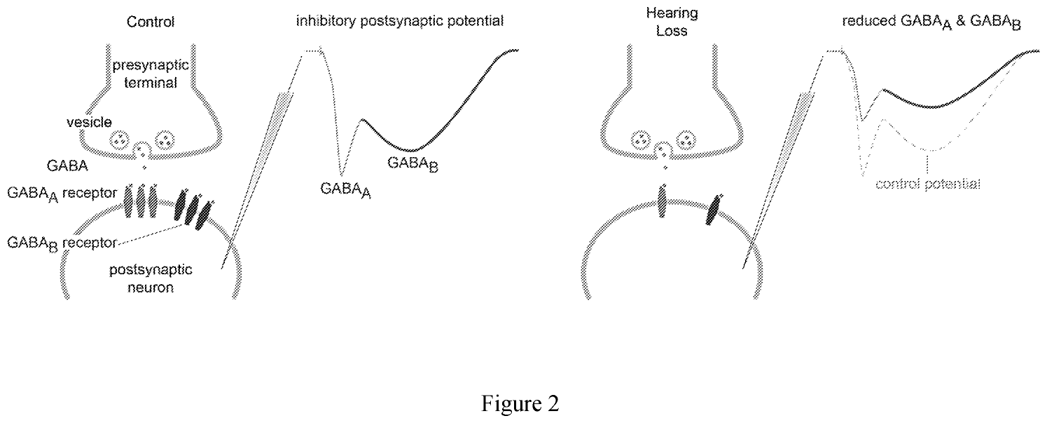

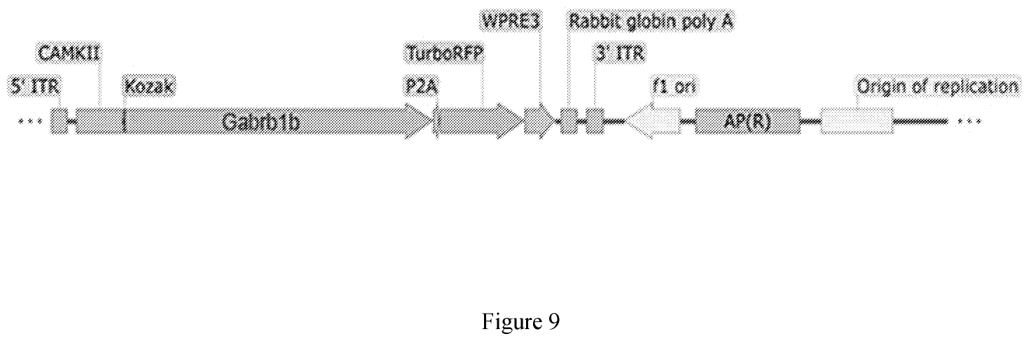

As discussed above, the disclosure relates to postsynaptic inhibition that is mediated by two types of GABA receptors, as illustrated in FIG. 1. As known in the art, and as shown in FIG. 1, GABA_{A} receptors are composed of 5 proteins, two of which are alpha subunits. GABA_{A} receptors mediate chloride entry into the neuron, thereby causing the membrane potential to become hyperpolarized (more negative). GABA_{B} receptors are composed of 2 proteins, one of which is the 1b subunit. GABA_{B} receptors are coupled to G-proteins and mediate potassium efflux from the neuron, thereby causing the membrane potential to become hyperpolarized. The present disclosure relates to these GABA receptors by providing two representative viral vectors to express the following inhibitory synapse receptors:
(1) Gabral (the GABA_{A} receptor alpha1 subunit): this subunit contributes to the pentameric receptor that mediates fast GABA-evoked postsynaptic inhibition. This construct is referred to as AAV1.CaMKII0.4.Gabra1.IRES.mCherry. WPRE.rBG. A schematic of this construct is provided in FIG. 9.
(2) Gabrb1b (the GABA_{B} receptor 1b subunit): this subunit contributes to the dimeric receptor that mediates slow GABA-evoked postsynaptic inhibition. This construct is referred to as AAV1.CaMKII0.4.Gabrb1b.P2A. TurboRFP.WPRE3.rBG. These vectors relate to a core pathology that occurs following developmental hearing loss, and many other developmental disorders that involve the reduction of GABA-mediated inhibition. One reason for this is that postsynaptic GABA receptor expression or membrane trafficking is deficient. FIG. 2 schematizes this effect and depicts a loss of both GABA_{A} and GABA_{B} receptors from the postsynaptic membrane.

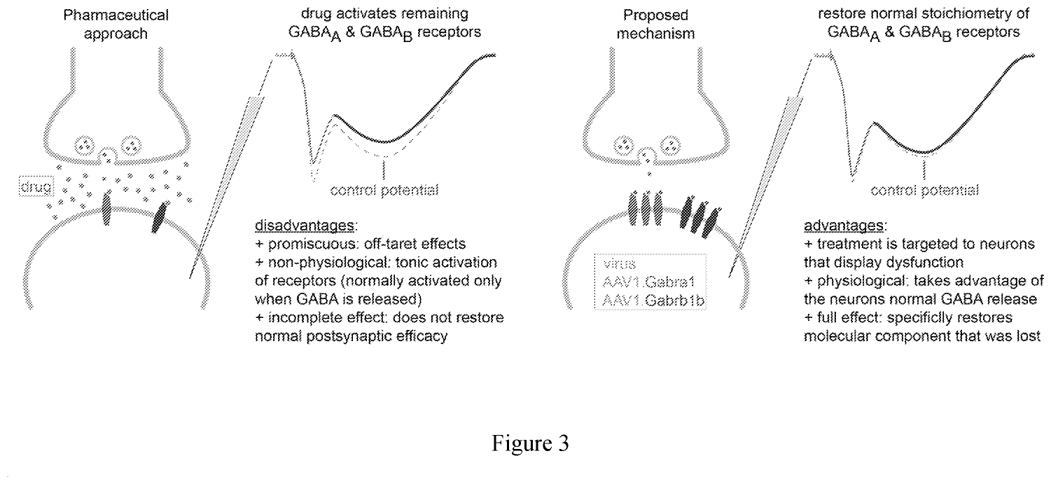

FIG. 3 illustrates the differences between traditional pharmaceutical approaches and that of the present disclosure. In this regard, many drugs have been developed that bind selectivity to each type of GABA receptor. In principle, one way to restore inhibition is to deliver drugs that bind to the remaining GABA_{A} and/or GABA_{B} receptors. However, this approach has disadvantages, as follows. The drugs cannot be restricted to the specific central neurons have become dysfunctional, and this can lead to off-target effects. The drugs bind to receptors and cause a constant, probabilistic opening. This is distinct from the way in which native GABA receptors operate. Normally, GABA is released from the presynaptic terminal, and it is only at this instant that GABA receptors are activated. The primary biological deficit-loss of postsynaptic GABA receptors—is unabated by the treatment.

In contrast, and without intending to be bound by any particular interpretation, the virally-mediated protein expression of this disclosure has at least the following advantages: The expression of the GABA receptor is localized to a small volume around the injection location, and can thus be targeted to any brain region exhibiting reduced inhibition. The expression of the GABA receptor can be further restricted to specific types of neurons in the injection site through the use of a specific gene promotor. In a non-limiting demonstration, the disclosure uses the CaMKII0.4 promoter to express the described receptors in excitatory neurons. The disclosure includes modifying this approach to express GABA receptors in another subset of neurons (such as a specific layer of cortex) or inhibitory neurons. When GABA receptors are expressed in neurons, they are trafficked to the postsynaptic membrane, where they are activated by the normal release of the neurotransmitter, GABA. This preserves the time-critical element of postsynaptic inhibition. The described approach directly treats the biological deficit such that lost receptors are replenished.

The following Examples are intended to illustrate but not limit aspects of this disclosure.

### Example 1

To demonstrate one embodiment of the disclosure, the Gabrblb expressing rAAV was expressed in auditory cortex. The protocol was as follows.

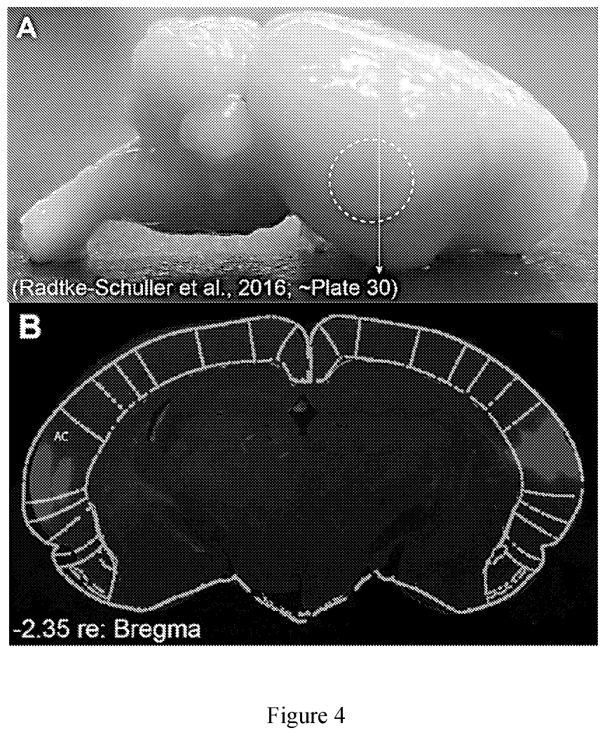

AAV1.CaMKII0.4.Gabrb1b.P2A. TurboRFP.WPRE3.rBG was injected bilaterally into auditory cortex of gerbils at postnatal day 23 (350 nL of virus at 2 nL/s). Animals survived until postnatal day 44, at which point they were perfused with fixative and the brains sectioned for fluorescent microscopy. The anatomical result is shown in FIG. 4. The co-expressed fluorophore, TurboRFP, was expressed at the injection site. FIG. 4, panel A shows a lateral view of the whole brain. Even under normal lighting conditions, TurboRFP is visible (dotted circle). FIG. 4, panel B shows a transverse tissue section from the brain shown in panel A. TurboRFP fluorescence is visible throughout the auditory cortex (AC) following 3 weeks of expression. TurboRFP is co-expressed with our gene of interest (Gabrb1b), which confirms the construct was introduced into the intended region of the brain (the Auditory Cortex).

### Example 2

This Example demonstrates that developmental hearing loss-induced perceptual deficits are rescued by the Gabrb 1b expressing viral construct.

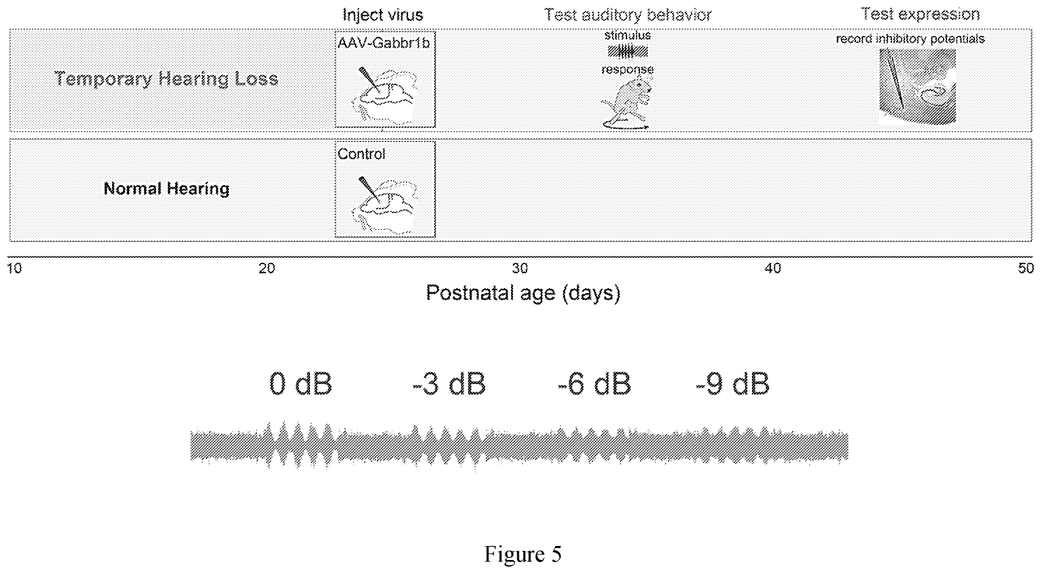

To obtain the described results, we induced temporary hearing loss from postnatal day 10 to 23 in gerbils. This is an animal model of the most common form of childhood hearing loss which is due to middle ear infections called otitis media). This coincides with the developmental time during which the auditory cortex is most vulnerable to the loss of experience in the gerbil. FIG. 5 shows the time line for induction of temporary hearing loss from postnatal days 10-23, the virus manipulation (AAV-Gabrb1b or control virus), and the outcome measures (behavior and physiology).

We used a behavioral paradigm where thirsty gerbils are trained to withdraw from a drinking spout when they hear a change from pure noise to amplitude modulated white noise or spectrally modulated while noise. All natural sounds, including human speech are largely composed of amplitude and spectral modulations. An amplitude modulated stimulus is illustrated in FIG. 5. Large negative values signify smaller modulations.

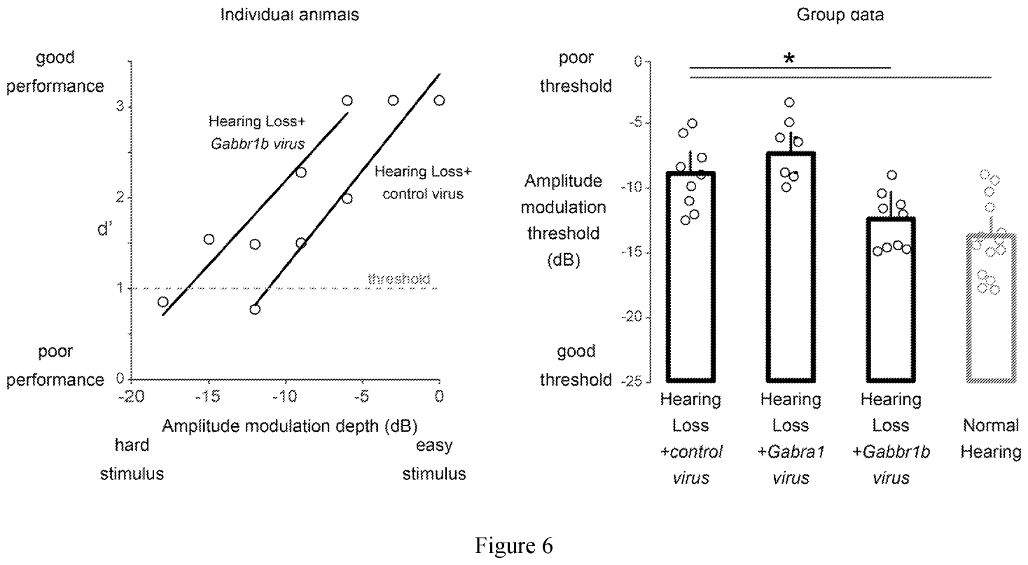

FIG. 6 shows the behavioral results from an experiment in which we demonstrate that developmental hearing loss impairs a perceptual skill (amplitude modulation detection), and the show that the representative construct expressing Gabrb 1b can rescue the hearing loss-induced deficit.

On the left of FIG. 6, example psychometric functions are shown for two individual animals that experienced Temporary Hearing loss. In one, we injected the Gabrb1b virus into the auditory cortex, and the other received an injection of control virus. The animal with Gabrb 1b virus performed better than the control in that its performance (measured as d′) was shifted to the left for each stimulus value. We extracted the threshold performance for each animal, defined as the amplitude modulation value at with d′=1.

In the graph (right) of FIG. 6, amplitude modulation detection thresholds are shown for each animal in our 3 treatment groups. The thresholds for animals with Hearing Loss were significantly poorer than those observed for animals treated with the Gabrb1b virus. In contrast, animals treated with the Gabral virus did not display improved performance. Therefore, expression of the Gabrblb virus is able to rescue performance on one perceptual task in animals that experienced Temporary Hearing Loss. For comparison, the thresholds of Normal Hearing animals from a previous experiment are shown. In FIG. 6 (left), AM depth psychometric functions for a HL-reared animal in which AC was injected with virus to express GFP or Gabrblb. In FIG. 6 (right), SM thresholds were significantly better in HL-reared animals that expressed Gabrb 1b, versus those that expressed GFP or Gabral. NH=normal hearing. These plots illustrate gerbil performance on a behavioral task which tests their ability to perceive amplitude modulation of white noise, illustrated below. This is sinusoidal modulation of the volume of white noise, at a rate of 6 Hz, and at varying degrees of modulation, where 0 dB indicates 100%, or easily detectable AM, and 9 dB indicates ˜35% modulation, which is somewhat harder to perceive. Perception of AM is important for speech perception in humans. Gerbils with intact hearing can generally detect AM down to ˜16 dB as indicated by the grey bar, while animals with developmental hearing loss (the earplug manipulation) can only detect AM down to about 12 dB. As can be seen from the figure, the Gabrb 1b virus restores perception of AM stimuli. The GFP virus is used as a control.

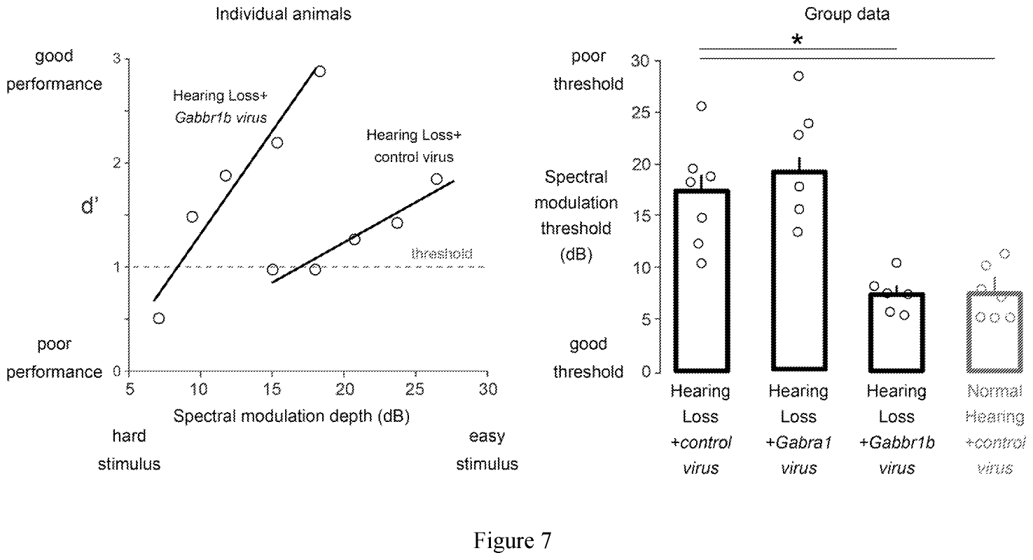

Next we tested whether the virus would restore perception of spectral modulation. Whereas amplitude modulation detection tests the animal's ability to perceive rapid changes in volume, spectral modulation tests an animal's ability to detect small changes to the frequency content of a stimulus. FIG. 7 shows the behavioral results from an experiment in which we demonstrate that developmental hearing loss impairs a perceptual skill (spectral modulation detection), and the show that that the representative construct encoding Gabrb1b can rescue the hearing loss-induced deficit.

Again, injection of the Gabrb1b virus into auditory cortex improved the performance of animals reared with Temporary Hearing loss, as compared to animals that received a control virus (. While Gabrb 1b did rescue perception, it did not restore it back to the performance of Normal Hearing animals.

### Example 3

This Example demonstrates that AAV1.CaMKII0.4.Gabra1.IRES.mCherry. WPRE.rBG leads to increased amplitude of GABA_{A} receptor mediated inhibitory potentials.

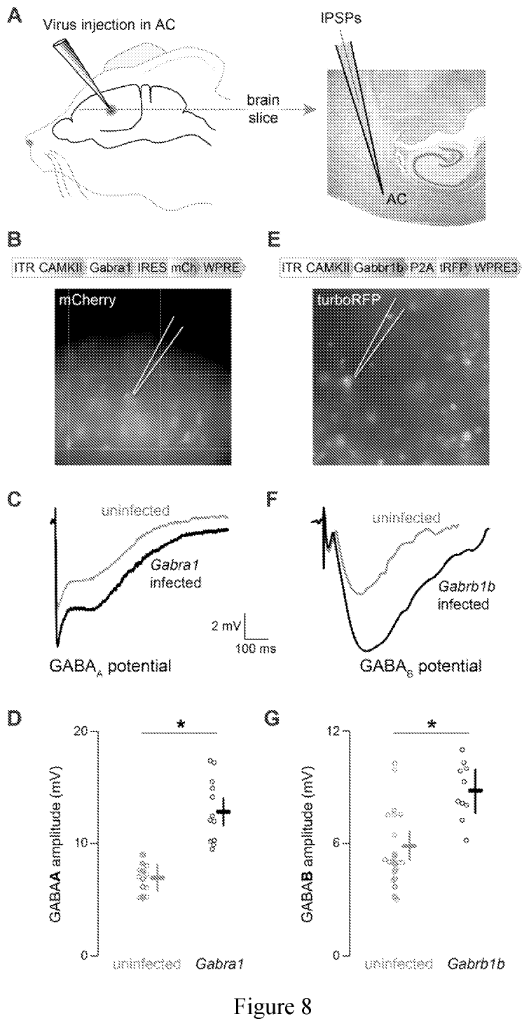

Experimental Protocol: To obtain a measure of increases in functional inhibition, we transfected auditory cortex neurons with the virus that expresses the GABA_{A} receptor protein, Gabra1. The results in FIG. 8 show a schematic of the procedure (panel A) in which virus is first injected in vivo, and following a survival period, brain slices are obtained for intracellular recordings. Using whole cell current clamp recordings, we then obtained inhibitory postsynaptic potentials from individual auditory cortex neurons that are either uninfected or express the virus (mCherry-positive).

Physiology Result: Two individual recordings are shown for an uninfected and infected neuron (FIG. 8, panels C and F), and show that the evoked IPSP is larger at a latency expected for GABA_{A} receptors. Summary data for uninfected and infected neurons show that GABA_{A} receptor potentials are significantly larger in infected auditory cortex neurons (FIG. 8, panels D and G).

### Example 4

This Example provides representative sequences that are encompassed by the disclosure and a description of making a described vector that is shown in FIG. 9. This Example provides non-human mammal and human sequences of components of the described vectors.

The following process was used to make the gerbil GABA Type B subunit 1b gene sequence. Using the mouse sequence (2535 BP including start codon; AF120255.1 Mus musculus gamma-aminobutyric acid B receptor 1b (Gabrb1b) mRNA, we ran BLAST against gerbil genome. This resulted in a 3275 bp gerbil sequence for which the partial sequence from 740 to 3134 aligned with the mouse sequence from 141 to 2535. The full gerbil sequence is available under accession number XM_021659989.1; predicted Meriones unguiculatus gamma-aminobutyric acid type B receptor subunit 1 (Gabrb1), transcript variant X2, mRNA). We added the first 140 bp from mouse sequence to the aligned portion of the gerbil sequence, thereby generating a 2535 bp sequence which is all gerbil sequence from bp 141 to 2535. Sequences encompassed by this disclosure are as follows.

Human gamma-aminobutyric acid receptor subunit alpha-1 precursor protein

(SEQ ID NO: 1)
MRKSPGLSDCLWAWILLLSTLTGRSYGQPSLQDELKDNTTVFTRILDRL
LDGYDNRLRPGLGERVTEVKTDIFVTSFGPVSDHDMEYTIDVFFRQSWK
DERLKFKGPMTVLRLNNLMASKIWTPDTFFHNGKKSVAHNMTMPNKLLR
ITEDGTLLYTMRLTVRAECPMHLEDFPMDAHACPLKFGSYAYTRAEVVY
EWTREPARSVVVAEDGSRLNQYDLLGQTVDSGIVQSSTGEYVVMTTHFH
LKRKIGYFVIQTYLPCIMTVILSQVSFWLNRESVPARTVFGVTTVLTMT
TLSISARNSLPKVAYATAMDWFIAVCYAFVFSALIEFATVNYFTKRGYA
WDGKSVVPEKPKKVKDPLIKKNNTYAPTATSYTPNLARGDPGLATIAKS
ATIEPKEVKPETKPPEPKKTFNSVSKIDRLSRIAFPLLFGIFNLVYWAT
YLNREPQLKAPTPHQ

Gerbil amino acid sequence for Gerbil GABA_{B} receptor 1b subunit (Gabrb1b)

(SEQ ID NO: 2)
MGPGAPCPPVGWPLPLLLVIAAGMAPVWASHSPHLPRPHPRVPPHPSSE
RRAVYIGALFPMSGGWPGGQACQPAVEMALEDVNSRRDILPDYELKLIH
HDSKCDPGQATKYLYELLYNDPIKIILMPGCSSVSTLVAEAARMWNLIV
LSYGSSSPALSNRQRFPTFFRTHPSATLHNPTRVKLFEKWGWKKIATIQ
QTTEVFTSTLDDLEERVKEAGIEITFRQSFFSDPAVPVKNLKRQDARII
VGLFYETEARKVFCEVYKERLFGKKYVWFLIGWYADNWFKTYDPSINCT
VDEMTEAVEGHITTEIVMLNPANTRSISNMTSQEFVEKLTKRLKRHPEE
TGGFQEAPLAYDAIWALALALNKTSGGGGRSGVRLEDFNYNNQTITDQI
YRAMNSSSFEGVSGHVVFDASGSRMAWTLIEQLQGGSYKKIGYYDSTKD
VLSWTKTDKWIGGSPPADQTLVIKTFRFLSQKLFISVSVLSSLGIVLAV
VCLSFNIYNSHVRYIQNSQPNLNNLTAVGCSLALAAVFPLGLDGYHIGK
SQFPFVCQARLWLLGLGFSLGYGSMFTKIWWVHTVFTKKEEKKEWRKTL
EPWKLYATVGLLVGMDVLTLAIWQIVDPLHRTIETFAKEEPKEDIDVSI
LPQLEHCSSKKMNTWLGIFYGYKGLLLLLGIFLAYETKSVSTEKINDHR
AVGMAIYNVAVLCLITAPVTMILSSQQDAAFAFASLAIVFSSYITLVVL
FVPKMRRLITRGEWQSEAQDTMKTGSSTNNNEEEKSRLLEKENRELEKI
IAEKEERVSELRHQLQSRQQLRSRRHPPTPPDPSGGLPRGPSEPPDRLS
CDGSRVHLLYK

Gerbil transcript for GABA_{B} receptor 1b subunit (gabrb1b)

(SEQ ID NO: 3)
ATGGGCCCGGGGGGACCCTGTACCCCGGTGGGGTGGCCGCTGCCTCTTC
TGCTGGTGATGGCGGCTGGGGTGGCTCCGGTGTGGGCCTCTCACTCCCC
TCATCTCCCGCGGCCTCACCCGAGGGTCCCCCCGCACCCCTCCTCAGAA
CGGCGTGCAGTGTACATCGGGGCGCTGTTTCCCATGAGCGGGGGCTGGC
CGGGGGGCCAGGCCTGCCAGCCCGCGGTGGAGATGGCGCTGGAGGACGT
GAACAGCCGCAGGGACATATTGCCGGACTACGAGCTCAAGCTTATCCAC
CACGACAGCAAGTGTGACCCAGGGCAAGCCACCAAGTACTTGTATGAAC
TACTCTACAATGACCCCATCAAGATTATCCTCATGCCTGGCTGCAGTTC
TGTCTCCACACTTGTGGCTGAGGCTGCCCGGATGTGGAACCTAATTGTG
CTCTCATATGGCTCCAGCTCACCAGCTTTGTCAAACCGACAGCGTTTTC
CAACATTCTTCCGAACACATCCGTCTGCCACACTCCACAATCCCACCCG
GGTGAAACTCTTTGAAAAGTGGGGCTGGAAGAAGATTGCCACCATCCAG
CAGACCACAGAGGTCTTCACTTCAACTCTGGATGACCTAGAGGAGCGAG
TGAAGGAGGCTGGGATTGAGATCACGTTCCGACAGAGTTTCTTCTCAGA
TCCAGCTGTGCCTGTTAAAAACCTGAAGCGTCAGGATGCTCGAATCATC
GTGGGACTTTTCTATGAGACTGAAGCCCGGAAAGTTTTTTGTGAGGTCT
ATAAGGAACGGCTCTTTGGGAAGAAGTATGTCTGGTTCCTCATTGGGTG
GTATGCTGACAACTGGTTCAAGACCTATGACCCATCAATCAACTGTACA
GTGGATGAAATGACCGAGGCAGTGGAAGGCCATATCACCACGGAGATCG
TCATGCTGAACCCTGCCAACACCCGAAGCATTTCCAACATGACATCCCA
GGAATTTGTGGAGAAACTAACCAAGAGGCTGAAGAGACACCCCGAGGAA
ACTGGAGGCTTCCAGGAGGCACCACTGGCCTATGATGCTATATGGGCCT
TGGCTCTGGCCTTGAATAAGACCTCTGGAGGAGGTGGCCGCTCGGGTGT
GCGCCTGGAGGACTTTAACTACAACAATCAGACCATTACAGACCAAATC
TACCGGGCCATGAACTCCTCCTCCTTTGAGGGTGTTTCTGGCCATGTGG
TCTTTGATGCCAGCGGCTCCCGGATGGCATGGACACTTATTGAGCAGTT
ACAGGGCGGCAGCTACAAGAAGATTGGCTACTACGACAGCACCAAGGAT
GTTCTCTCCTGGACCAAAACAGATAAGTGGATTGGAGGGTCTCCTCCAG
CTGACCAGACCTTGGTCATCAAGACATTCCGTTTCCTGTCACAGAAACT
CTTTATCTCCGTCTCAGTTCTCTCCAGCCTGGGCATTGTTCTTGCTGTT
GTCTGTCTGTCCTTTAACATCTACAACTCCCACGTTCGTTATATCCAGA
ACTCTCAGCCCAACCTGAACAATCTGACTGCCGTGGGCTGCTCACTGGC
ACTAGCTGCTGTCTTTCCCCTTGGGCTGGATGGTTACCACATAGGGAAA
AGCCAGTTCCCATTTGTCTGCCAGGCCCGACTTTGGCTCTTGGGCTTGG
GCTTTAGTCTGGGTTATGGCTCTATGTTCACCAAGATCTGGTGGGTCCA
CACAGTCTTCACAAAGAAGGAGGAGAAGAAGGAATGGAGGAAGACCCTA
GAACCTTGGAAACTGTATGCCACTGTAGGCTTGCTGGTGGGAATGGATG
TCCTGACTCTTGCCATCTGGCAGATTGTGGACCCCTTGCACCGAACCAT
TGAGACTTTTGCCAAGGAGGAGCCAAAGGAAGACATTGATGTCTCCATC
CTGCCCCAGCTGGAGCACTGCAGCTCCAAGAAGATGAATACATGGCTTG
GCATTTTCTATGGTTACAAGGGGCTGCTGCTGCTGCTGGGAATCTTTCT
TGCCTATGAGACTAAAAGCGTGTCCACTGAAAAGATCAACGACCACAGG
GCTGTGGGTATGGCCATCTACAATGTTGCGGTCCTGTGTCTCATCACTG
CTCCTGTGACCATGATTCTTTCCAGCCAGCAGGACGCAGCCTTCGCCTT
CGCCTCTCTGGCCATCGTGTTCTCTTCCTACATTACGCTGGTTGTGCTC
TTTGTGCCCAAGATGCGCAGGTTGATCACTCGGGGGGAGTGGCAGTCCG
AAGCGCAAGACACCATGAAAACGGGGTCGTCCACCAACAACAACGAGGA
AGAGAAGTCCCGGCTGTTGGAGAAAGAAAACCGTGAATTGGAAAAGATC
ATTGCTGAGAAAGAGGAGCGGGTCTCGGAACTGCGCCATCAGCTCCAGT
CTCGGCAGCAGCTGCGCTCACGGCGCCACCCTCCAACACCCCCAGACCC
CTCTGGGGGCCTTCCCAGGGGCCCCTCGGAGCCCCCTGACCGGCTGAGC
TGTGATGGGAGTCGAGTGCATTTGCTGTACAAGTGA

Homo sapiens gamma-aminobutyric acid receptor subunit beta-1 precursor protein

(SEQ ID NO: 4)
MGPGAPFARVGWPLPLLVVMAAGVAPVWASHSPHLPRPHSRVPPHPSSE
RRAVYIGALFPMSGGWPGGQACQPAVEMALEDVNSRRDILPDYELKLIH
HDSKCDPGQATKYLYELLYNDPIKIILMPGCSSVSTLVAEAARMWNLIV
LSYGSSSPALSNRQRFPTFFRTHPSATLHNPTRVKLFEKWGWKKIATIQ
QTTEVFTSTLDDLEERVKEAGIEITFRQSFFSDPAVPVKNLKRQDARII
VGLFYETEARKVFCEVYKERLFGKKYVWFLIGWYADNWFKIYDPSINCT
VDEMTEAVEGHITTEIVMLNPANTRSISNMTSQEFVEKLTKRLKRHPEE
TGGFQEAPLAYDAIWALALALNKTSGGGGRSGVRLEDFNYNNQTITDQI
YRAMNSSSFEGVSGHVVFDASGSRMAWTLIEQLQGGSYKKIGYYDSTKD
DLSWSKTDKWIGGSPPADQTLVIKTFRFLSQKLFISVSVLSSLGIVLAV
VCLSFNIYNSHVRYIQNSQPNLNNLTAVGCSLALAAVFPLGLDGYHIGR
NQFPFVCQARLWLLGLGFSLGYGSMFTKIWWVHTVFTKKEEKKEWRKTL
EPWKLYATVGLLVGMDVLTLAIWQIVDPLHRTIETFAKEEPKEDIDVSI
LPQLEHCSSRKMNTWLGIFYGYKGLLLLLGIFLAYETKSVSTEKINDHR
AVGMAIYNVAVLCLITAPVTMILSSQQDAAFAFASLAIVFSSYITLVVL
FVPKMRRLITRGEWQSEAQDTMKTGSSTNNNEEEKSRLLEKENRELEKI
IAEKEERVSELRHQLQSRQQLRSRRHPPTPPEPSGGLPRGPPEPPDRLS
CDGSRVHLLYK

CaMKII0.4 promoter

The promoter sequence used in the described vector is a mouse α-calcium/calmodulin-dependent protein kinase II (α-CaMKII), 0.4-kb PCR fragment. The sequence of this promoter is known, for example, from FIG. 3 in Sunyer T, Sahyoun N (1990) Sequence analysis and DNA-protein interactions within the 5′ flanking region of the Ca2+/calmodulin-dependent protein kinase II alpha-subunit gene. PNAS 87 (1): 278-82. doi: 10.1073/pnas.87.1.278, the disclosure of which is incorporated herein by reference.

While the disclosure has been particularly shown and described with reference to specific embodiments, it should be understood by those having skill in the art that various changes in form and detail may be made therein without departing from the spirit and scope of the present disclosure as disclosed herein.

1. A recombinant polynucleotide for use in reducing central nervous system sequelae of hearing loss, or improving hearing in an individual in need thereof, the recombinant polynucleotide comprising a promoter that is operably linked to a sequence encoding a Gamma-aminobutyric acid (GABA) receptor.

2. The recombinant polynucleotide of claim 1, wherein the promoter selectively drives expression of the GABA receptor in neuronal cells, said neuronal cells optionally being cortical pyramidal neurons.

3. The recombinant polynucleotide of claim 1, wherein the GABA receptor comprises a GABA_{A} receptor alpha 1 subunit or GABAR receptor 1b subunit.

4. The recombinant polynucleotide of claim 1, wherein the promoter comprises a CaMKII promoter.

5. The recombinant polynucleotide of claim 4, comprising the sequence encoding the GABA_{A} receptor alpha 1 subunit.

6. The recombinant polynucleotide of claim 4, comprising the sequence encoding the [or] GABA_{B} receptor 1b subunit.

7. The recombinant polynucleotide of claim 6, wherein the recombinant polynucleotide is comprised by an expression vector.

8. The recombinant polynucleotide of claim 7, wherein the expression vector comprises a viral vector.

9. The recombinant polynucleotide of claim 8, wherein the expression vector comprises an adenoviral expression vector.

10. Viral particles comprising the recombinant polynucleotide of claim 1.

11. A method comprising administering to an individual in need thereof an effective amount of viral particles of claim 10 such that the sequelae of hearing loss in the individual is inhibited, or hearing of the individual is improved.

12. The method of claim 11, wherein the GABA receptor is expressed in neuronal cells present in the auditory cortex of the individual.

13. The method of claim 11, wherein the viral particles comprise a combination of viral particles that separately express the GABA_{A} receptor alpha 1 and the GABAR receptor 1b subunits.

14. The method of claim 11, wherein the viral particles comprise adenoviral particles, and wherein optionally the adenoviral particles are introduced into the brain of the individual such that the GABA receptor is expressed in neuronal cells present in the auditory cortex of the individual.

15. The method of claim 11, wherein the hearing of the individual is improved.

16. The method of claim 15, wherein hearing improvement comprises improved sensitivity to amplitude modulation, improved sensitivity to spectral modulation, or a combination thereof, including natural sounds such as human speech that are composed of amplitude and spectral modulations.

17. A method comprising introducing into neuronal cells a recombinant polynucleotide of claim 1 such that the neuronal cells express the GABA receptor, wherein the expression of the GABA receptor is driven by the promoter.

18. The method of claim 17, wherein the neuronal cells are within an individual.

19. The method of claim 17, wherein the neuronal cells are cortical pyramidal neurons.

20. The method of claim 19, wherein expression of the GABA receptor is restricted to the cortical pyramidal neurons.
